(19) United States
(12) Patent Application Publication
(10) Pub. No.: US 2004/0138827 A1
(43) Pub. Date: Jul. 15, 2004

(54) Integrated, intelligent micro-instrumentation platform for protein crystallization

(75) Inventors: Guann-Pyng Li, Irvine, CA (US); Mark Bachman, Irvine, CA (US); Abraham P. Lee, Irvine, CA (US); Nancy L. Albritton, Irvine, CA (US); Brigette Schobert, Irvine, CA (US); Noo Li Jeon, Irvine, CA (US); Patrick Coffey, Irvine, CA (US); Janos K. Lanyi, Irvine, CA (US); Liang Li Wu, Irvine, CA (US)

Correspondence Address: ORRICK, HERRINGTON & SUTCLIFFE, LLP, 4 PARK PLAZA, SUITE 1600, IRVINE, CA, 92614-2558, US

(73) Assignee: The Regents of the University of California

(21) Appl. No.: 10/669,231

(22) Filed: Sep. 23, 2003

Related U.S. Application Data
(60) Provisional application No. 60/413,122, filed on Sep. 23, 2002.

(51) Int. Cl. G06F 19/00; C07K 17/00

(52) U.S. Cl. 702/27; 530/333

(57) ABSTRACT

Systems and methods that facilitate crystallization studies through the use of a system of microfluidics, sensors, actuators, and computer instrumentation to enable automated microcrystallization experiments using feedback assisted control and data from a protein crystallization or solubility database. In a preferred embodiment, the crystallization system comprises a database containing quantitative information about the chemical and physical properties under various thermodynamics conditions of the molecule or compounds of interest and a feedback assisted, dynamically controlled chemical system. The chemical system includes a controller that accesses the database and can monitor and control the state of one or more experiments, a software program that uses database information, sensory information, historical information and the like to change the conditions of the experiment, and a crystallization platform that allows computer control and sensing of one or more chemical experiments.

### FIELD OF THE INVENTION

The present invention relates generally to crystallization of proteins and other crystallizable media and, more particularly, to an integrated, intelligent microinstrumentation platform that facilitates high performance, high throughput crystallization of proteins and other crystallizable media.

### BACKGROUND INFORMATION

The most common methods of crystallization involve the placement of the sample to be crystallized in a small container. The sample is then allowed to change its internal chemical and thermodynamic state through the controlled removal of solvents (typically water) or change in concentration of solutes (e.g., salt). Typically the rate of exchange is controlled by an ion specific membrane or gas gap between two solutions of differing solute concentrations. Ion concentration in the sample is thus modified by the slow exchange of molecules that occurs through osmosis from one solution to the other. The conditions for crystal growth are difficult to predict and one usually finds the proper conditions by trial and error, an expensive and time consuming process.

To increase throughput, and efficiency, large scale crystallization experiments are often performed on many containers at once, each containing samples with different initial chemical conditions, typically salt concentration and pH. To prepare many experiments, one must pipette many different reagents in many different containers. This is an expensive and time consuming operation. To increase throughput, robotic pipette dispensers are often employed. However, these systems are expensive and clumsy, and use large amounts of crystallization sample (often a rare and expensive material).

To reduce the sample size, several companies have begun to develop microfluidics based crystallization devices. There relatively simple devices use micromachined fluidic channels to meter out tiny quantities of reagent over many small crystallization chambers, thus reducing sample usage while enabling large numbers of crystallization conditions to be explored. While this offers some advantages over traditional and robotic technology (high throughput, low sample consumption), the technology is different only by the method that the reagents are metered and delivered.

All methods in the current art use the same basic strategy to crystallize: set the initial conditions, wait, and check the results later. Very little technology in the current art uses much more than this to control crystallization. Thus the experimenter is left with few parameters to explore when trying to succeed in growing high quality crystals

### SUMMARY

The present invention is directed to systems and methods that facilitate crystallization studies through the use of a system of microfluidics, sensors, actuators, and computer instrumentation to enable automated microcrystallization experiments using feedback assisted control and data from a protein crystallization or solubility database. In a preferred embodiment, the crystallization system comprises a database containing quantitative information about the chemical and physical properties under various thermodynamics conditions of the molecule or compounds of interest and a feedback assisted, dynamically controlled chemical system. The chemical system includes a controller that accesses the database and can monitor and control the state of one or more experiments, a software program that uses database information, sensory information, historical information and the like to change the conditions of the experiment, and a crystallization platform that allows computer control and sensing of one or more chemical experiments. The platform preferably senses many different conditions of the experiment(s), including, but not limited to, temperature, volume, pH, concentration, refractive index, color, turbidity, absorbance, fluorescence, conductivity, viscosity, density, and the like. The platform preferably changes the chemical or physical state of the experiment(s) by many different mechanisms, including controlling the temperature, the humidity, the pH, the precipitant concentration, and the like to achieve crystallization.

The crystallization system preferably integrates multiple sensors and actuators with a microfluidics system to allow an experimenter to modify many parameters in a crystallization test which may affect the outcome of the experiment. These parameters may be modified at any time during the crystallization experiment. The use of a computer-based control system or controller to monitor and change the system in a preprogrammed manner, simplifies the effort and enables highly sophisticated crystallization experiments to be designed and performed consistently.

Other systems, methods, features and advantages of the invention will be or will become apparent to one with skill in the art upon examination of the following figures and detailed description.

### BRIEF DESCRIPTION OF THE FIGURES

The details of the invention, including fabrication, structure and operation, may be gleaned in part by study of the accompanying figures, in which like reference numerals refer to like parts. The components in the figures are not necessarily to scale, emphasis instead being placed upon illustrating the principles of the invention. Moreover, all illustrations are intended to convey concepts, where relative sizes, shapes and other detailed attributes may be illustrated schematically rather than literally or precisely.

FIG. 1 is a schematic of a crystallization system in accordance with the present invention.

FIG. 2 is an exemplary phase diagram depicting a graphic representation of the solubility of a protein versus precipitant concentration.

FIG. 3 is a side view of a multilayer crystallization platform of the present invention including a mid-layer crystallization cassette and showing electrical and optical components across the layers.

FIG. 4 is a schematic of a fluidic layout for microfluidic multiplexing in a crystallization cassette.

FIG. 5 is a schematic (underside) of a fluidic layout for microfluidic multiplexing in a 2×3 fluidic cassette.

FIG. 6 is a schematic of a gas line layout of a crystallization cassette for vapor control within a crystallization chamber.

FIG. 7 is a schematic depicting the operation of a magneto-hydrodynamic micropump.

FIG. 8 is a perspective view of a crystallization cassette showing crystallization chambers, vapor lanes and fluid lanes.

FIG. 9 is a schematic of the optics for a dynamic light scattering system.

FIG. 10 is a schematic of a vapor control chip.

FIG. 11 depicts a side view of the vapor control chip shown in FIG. 10.

### DETAILED DESCRIPTION

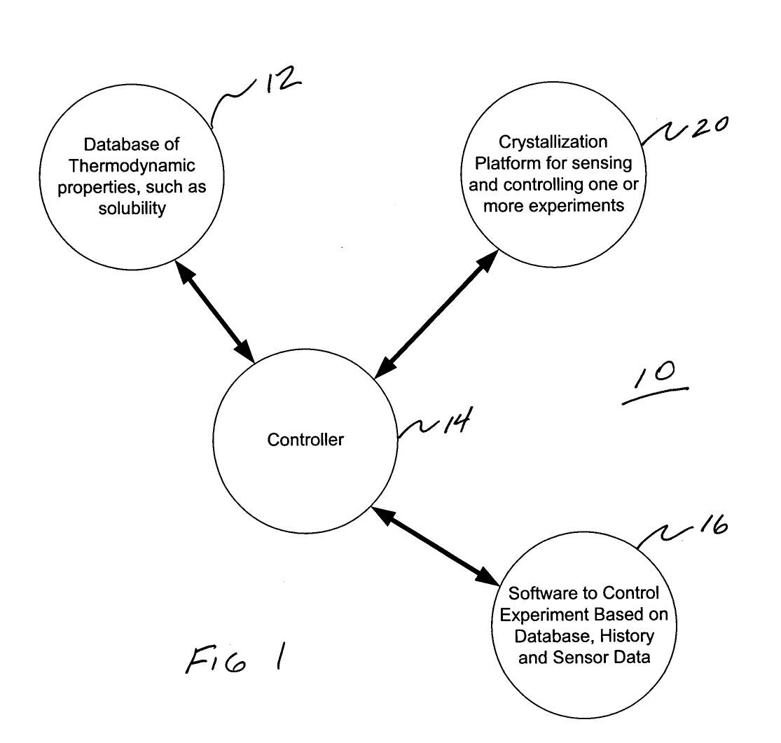

The systems and methods described herein facilitate more efficient, higher quality protein and other compound crystallization studies through the utilization of a system of microfluidics, sensors, actuators, and computer instrumentation to enable automated microcrystallization experiments using feedback assisted control and data from a protein crystallization or solubility database. As depicted in FIG. 1, the crystallization system 10 of the present invention comprises a database 12 containing quantitative information about the chemical and physical properties under various thermodynamics conditions of the molecule or compounds of interest and a feedback assisted, dynamically controlled chemical system comprising a controller 14 such as a computer or the like that accesses the database and can monitor and control the state of one or more experiments, a software program 16 that uses database information, sensory information, historical information and the like to change the conditions of the experiment, and a crystallization platform 20 that allows computer control and sensing of one or more chemical experiments. The platform preferably senses many different conditions of the experiment(s), including, but not limited to, temperature, volume, pH, concentration, refractive index, color, turbidity, absorbance, fluorescence, conductivity, viscosity, density, and the like. The platform preferably changes the chemical or physical state of the experiment(s) by many different mechanisms, including controlling the temperature, the humidity, the pH, the precipitant concentration, and the like to achieve crystallization.

The crystallization system 10 of the present preferably integrates multiple sensors and actuators with a microfluidics system to allow an experimenter to modify many parameters in a crystallization test which may affect the outcome of the experiment. These parameters may be modified at any time during the crystallization experiment. The use of a computer-based control system or controller 14 of the present invention to monitor and change the system in a preprogrammed manner, simplifies the effort and enables highly sophisticated crystallization experiments to be designed and performed consistently.

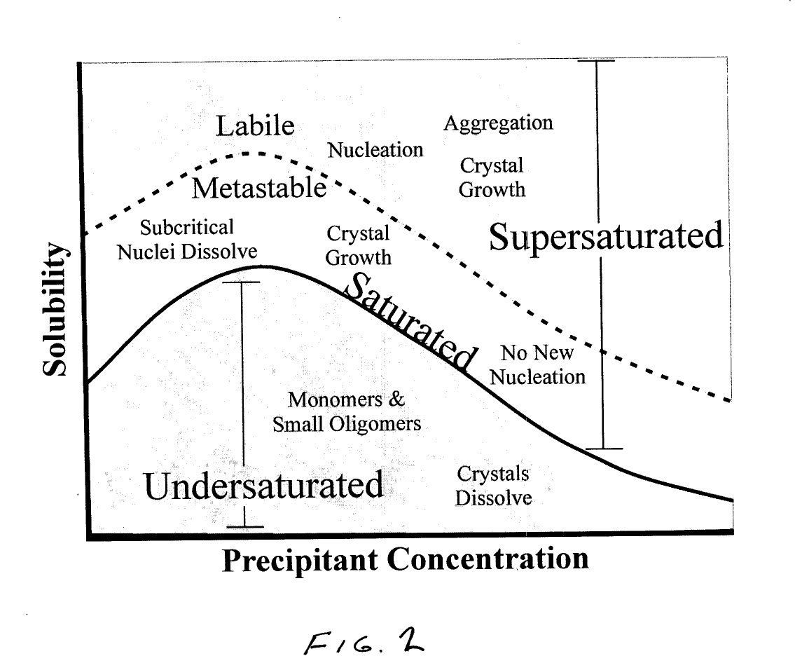

Crystallization is basically a two-step process occurring in a supersaturated solution. The first step is the spontaneous formation of nuclei in solution followed by the growth of the crystal. This process is influenced by a large number of factors which crystallographers manipulate in attempting to produce adequate crystals for X-ray diffraction. A phase diagram is a graphic representation of the solubility of a protein vs. one of these influences, such as temperature, pH, salt (precipitant) concentration, etc. An example of a phase diagram showing the solubility of a protein vs. precipitant concentration is provided in FIG. 2. The phase diagram is characterized by two regions: 1) the unsaturated region where the probability of nuclei formation is low and solid phase protein dissolves and 2) the supersaturated region which is further divided into metastable and labile regions. In the labile region the protein exists in a state of extreme supersaturation and the probability of nuclei formation is high; however, if conditions remain in this region, the protein is likely to form large numbers of nuclei resulting in useless microcrystals or amorphous precipitate. At lesser values of supersaturation (the metastable region) the probability of spontaneous formation of nuclei is low, but crystals grow well. Since the goal is to grow a minimum number of crystals of the largest size possible, it is desirable to set conditions to initiate nuclei formation in the labile region and at the appropriate time manipulate selected variables to form a metastable condition for promoting crystal growth, i.e. rationally exploit the phase diagrams. The limitation to attaining this ideal is that each protein has its own unique solubility profile. Because it is not possible to predict complete phase diagrams nor has it been practical in the past to generate them experimentally because the number of variables is so great, protein chemists have had to utilize empirical methods that are evolved from trial and error in hopes of achieving crystal formation for each protein. Due to the limited technology available, only rudimentary changes in conditions affecting the phase diagram have been possible for promoting crystal growth. Further, because of the lack of adequate feedback monitoring during the crystallization process, it is difficult for the protein chemists to perform this feat rationally. The feedback assisted, dynamically controlled chemical system 10 of the present invention, with its use of data from a solubility or thermodynamic database 12 corresponding to the protein or compound of interest, makes it possible to rationally exploit the solubility profile of a protein or compound of interest and more efficiently achieve crystallization.

Mapping the phase diagrams or solubility profiles of a protein and, thus, providing a solubility or thermodynamic database 12 can be accomplished by performing a large number of experiments that vary different thermodynamic properties (e.g., temperature and concentration) and monitor chemical or physical properties of the solution under those conditions. These experiments may be performed using conventional techniques including set up by hand or by robot, or by other more efficient techniques such as generating solution drops in oil as described in “Method and Apparatus For Continuously Varying The Chemical Composition Of Small Droplets,” U.S. Provisional Application No. 60/491207, filed Jul. 29, 2003, which is incorporated herein by reference as if set forth in full.

The controller 14 of the present invention comprises a computer-based control system capable of accessing the database 12 and control the experiment conducted on the crystallization platform 20. The controller 14 may be a dedicated device having a CPU and non-volatile memory, and adapted to receive and operably couple to a crystallization platform 20, preferably in the form of micro-cassette or chip. Alternatively, the controller 14 may be a computer to which the crystallization platform 20 couples through the computer's communication ports such as the parallel, serial or USB ports. In a preferred embodiment, the controller 14 preferably accesses the data contained in the thermodynamic database 12, accesses sensor data of the experiment(s) from sensors associated with the crystallization platform 20, runs the software 16, which is preferably stored along with the database 12 in the controller's 12 non-volatile memory, to determine how to change the experimental conditions, and direct the crystallization platform 20 to change the physical or chemical conditions of experiment(s).

For control of each experiment in the system 10, the software 16 preferably uses any or all of (1) the database of thermodynamic properties, (2) the sensory data collected by the computer, (3) historical information about the state of the experiment, (4) user supplied data about the experiment, and (5) information about other experiments under observation. In order to determine how to adjust the chemical conditions of the experiment, if at all, the software 16 preferably incorporates one or more of the following: statistics-based algorithms, pattern recognition schemes, maximum likelihood analysis, trend analysis, and the like. In addition, the software 16 may be adapted to defer to a human user in certain instances to direct the course of the experiment.

For sensing and controlling one or more experiments, a variety of different platform 20 configurations are acceptable. The crystallization platform 20, however, preferably comprises the following major components:

(1) a microfluidics system (microfluidics “card”) consisting of small (sub millimeter) channels, manifolds and wells designed to transport and manage fluids (gas and liquid;

(2) a system of sensors, either integrated directly in the microfluidics card or external to the card, designed to measure various physical, electrical, optical and chemical properties of the fluids in the card; and

(3) a system of actuators, either integrated directly in the microfluidics card or external to the card, designed to control the physical, electrical, optical or chemical state of the fluids in the card.

The platform 20 preferably integrates these components into a single instrument, designed specifically to perform crystallization experiments. The platform 20 may support one experiment at a time (a single crystallization chamber) or a large number of experiments simultaneously.

The controller or computer 20 and associated instrumentation preferably monitors the sensors, change the state of the actuators, control the crystallization experiments, and provide an interface to researchers who use the tool. The state variables that are monitored by the platform 20 preferably include, but are not limited to: pH, temperature, pressure, flow velocity, turbidity, degree of crystallization, spectral absorbance, fluorescence, birefringence, electrical resistance, electric impedance, electric field, and electric current. The experimental parameters that are controlled by the controller 14 and platform 20 preferably include, but are not limited to: temperature, pressure, flow, volume geometry, micro-agitation, initial pH, initial sample concentration and composition, initial seeding conditions, molecular exchange barrier, electromagnetic field, electric current and light intensity.

In operation, the platform 20 preferably enables precise control and monitoring of micro-experiments performed on the small microfluidic array cassette. For each micro-experiment, control will be afforded over the initial chemical conditions, temperature profiles, and even the chemical concentrations during the course of the experiment. This will have the dramatic effect of enabling complete control over the phase space of the crystallization experiments—a revolutionary capability for protein crystallization.

A schematic of one embodiment of the crystallization platform 20 of the present invention is shown in FIG. 3. As depicted, the platform 20 includes three layers designed to work together. The bottom layer 21, is preferably constructed of printed circuit board and other materials, and includes the electrical and photonic systems needed to control temperature and provide coherent and non-coherent light sources for static and dynamic light scattering (SLS; DLS) and imaging applications. The central layer 22, is preferably constructed of transparent plastic such as acrylic, polycarbonate, or the like, and includes the fluid handling and vapor handling systems needed to control the chemistry within the protein precursor solutions (“mother liquors”) and provide environmental chambers 30 for crystallization. The top layer 23 preferably comprises imaging optics and CMOS or CCD arrays needed for imaging crystals formed during the crystallization process and collecting light from DSL experiments. Although the subsystems are designed to work as an integrated system, they can also function independently. For example, one could pipette crystallization volumes within the chambers 30 and still utilize the temperature control and imaging systems.

Photonic and electrical subsystems: The base layer 21 includes electrical systems designed to provide temperature control by providing current across electrical contacts 28 on the printed circuit board 27 and central layer 22 to strategically placed thermistors 29. By passing current through the thermistors 29 (in pulse mode), the thermistors will act as heaters, heating up the small volume crystallization chambers 30. After waiting several seconds for the thermistor temperature to equilibrate with the chamber environment, the thermistor resistances may be measured with low current to determine the temperature of the chamber 30. Thermistors, which have very large temperature coefficients, may be used to maintain temperatures with 0.1 C. precision. The entire assembly is preferably placed on a cold plate (thermoelectric cooler) to provide an efficient heat sink. Thus, temperatures above and below thermal ambient may be achieved for each chamber 30. Furthermore, by maintaining suitable distance between each chamber 30, each crystallization experiment may be operated with a different temperature from its neighbors.

Photonic devices are preferably positioned under the printed circuit board 27. The two primary photonic devices are laser diodes 26 and light emitting diodes (LEDs) 24. The LEDs 24 are positioned to provide basic illumination for monochromatic imaging. Their light may pass through a beam splitter 25 and be reflected up through the crystallization chamber 30. Monochromatic imaging (single color) reduces the requirement for chromatic dispersion-correcting optics. The laser light 35 may be directed through the same chamber 30 by the beam splitter 25 to provide coherent light for DLS measurements.

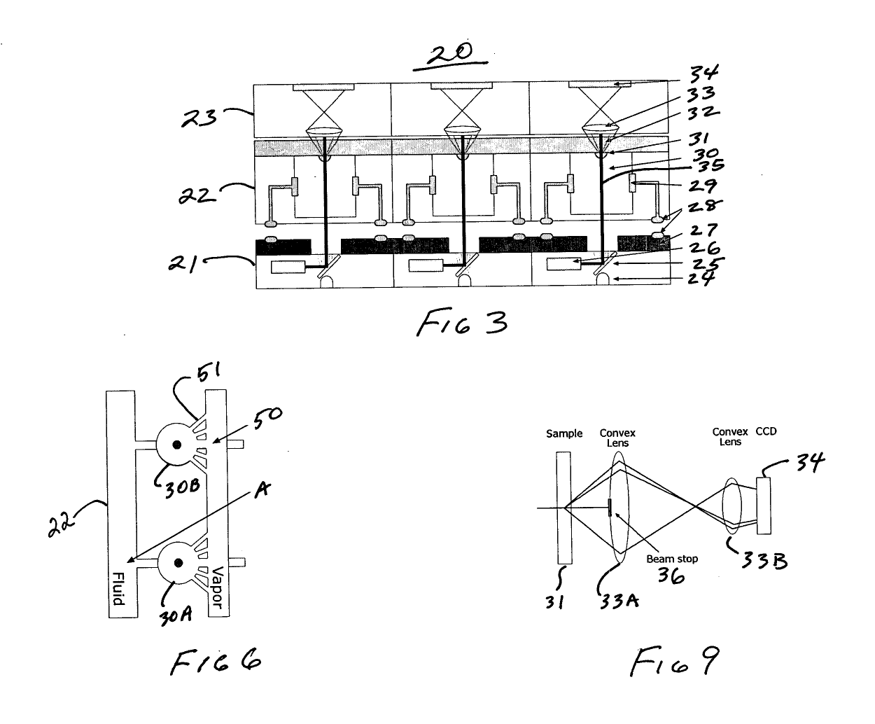

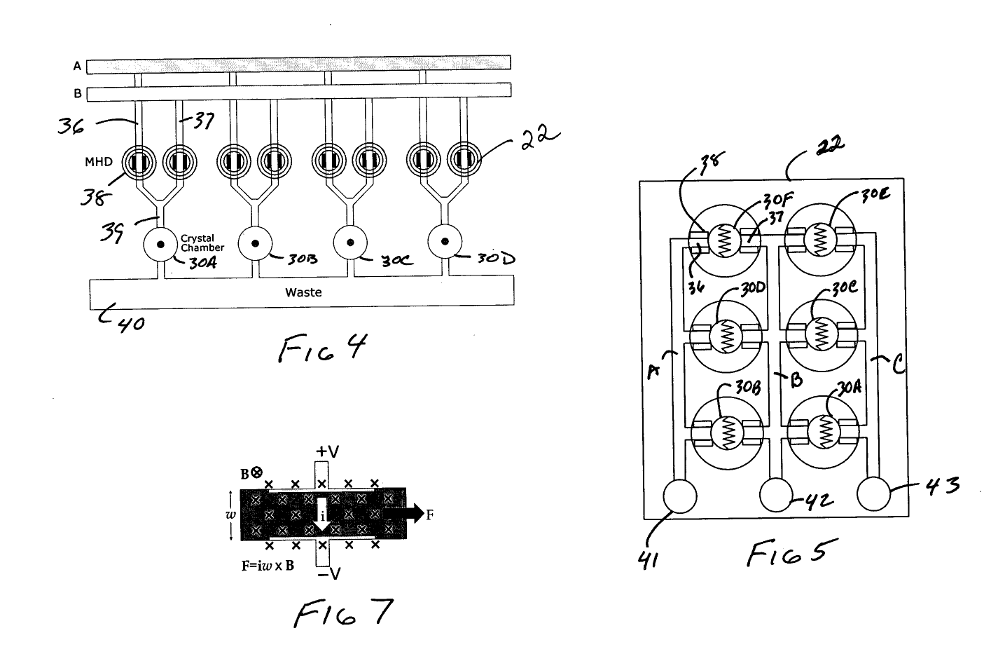

Fluidic and vapor subsystems: FIGS. 4 and 5 demonstrates the principle of fluidic multiplexing and FIG. 6 shows the concept of vapor control. Fluidic multiplexing allows multiple concentrations of mixtures to be generated from two or more stock solutions in fluid channels or lanes A, B and C. As shown in FIG. 5, the stock solutions are stored in wells 41, 42 and 43 formed in the cassette 22. Magneto-hydrodynamic pumps (MHD) 38 are used to generate fluid flows of different flow rates through the inlet channels 36 and 37 into a single mixing channel 39, followed by a metering of a small volume sample into a crystallization chamber 30. The remainder of the mixed buffer may be sent to more crystallization chambers, or to a waste collection line 40.

Magneto-hydrodynamic pumping uses an alternating magnetic field coupled with an alternating current passing across a fluidic line to generate flow by the resulting Lorentz force, as shown in FIG. 7. When an AC current of sufficiently high frequency is passed through an electrolytic solution, the electrochemical reactions at the electrodes are reversed rapidly enough that bubbles never form and no electrode degradation occurs. The time-averaged Lorentz force not only depends on the current amplitude or the magnetic field amplitude but also depends on the phase of the magnetic field, relative to the electrode current. The ability to control the phase difference enables the control of not only the flow speeds but also the flow direction. At 0° phase, the resulting force is positive and corresponds to a flow in one direction. At 180°, the resulting force is negative and corresponds to flow in the opposite direction. At 90° phase, there is no net flow.

Vapor control is afforded through the use of vapor control lines 50 that feed into every crystallization chamber 30A and 30B through vapor finger channels 51, as shown in FIG. 6. By controlling the surrounding humidity in the crystallization chamber 30A, 30B, water may be induced to flow into or out of the mother liquor (condensation or evaporation). Due to the small scale of the system, humidity in a crystallization chamber 30A, 30B may be quickly changed by changing the water content of the vapor control lines 50. Humidity of the vapor control lines 50 is programmed by controlling the flow of a dry, inert gas passing a water supply. Multiple crystallization chambers may be fed by a single line 50 (for parallel control), or each chamber may receive its own vapor control line.

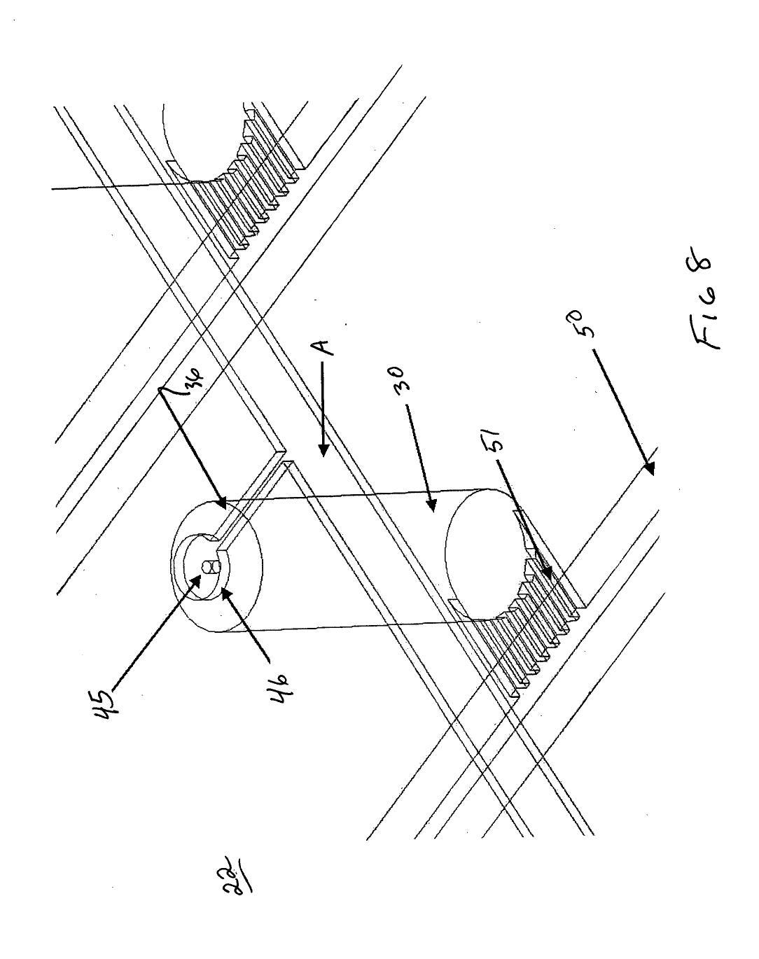

FIG. 8 provides a perspective view of the cassette 22 showing the relative orientation of the fluid lines, vapor lines and crystallization chambers. As depicted, a fluid line A feeds a crystallization chamber 30 via injection line 36 through a membrane valve 46 having an inlet 45. On the other side of the cassette 22 and chamber 30, a vapor line 50 is in communication with the chamber 30 via finger channels 51.

Optical imaging subsystems: Imaging and DLS provide feedback about the status of crystallization for the crystallographer performing the experiments or to the computer controlled system 14 attempting to automatically optimize crystallization conditions. Imaging may be accomplished through traditional means at low cost, by exploiting CMOS sensor arrays. In this embodiment, a single monochromatic light source 24 (e.g., a red or green LED) is utilized for imaging purposes. This provides two key advantages: first, simpler optics may be employed since compensation for chromatic aberration is not required; second, CMOS sensors can provide higher resolution under monochromatic light since all sensors may be used to image the device. Traditionally, a CMOS sensor uses four sensors per pixel to accomplish a RGB color image.

DLS is commonly used to study protein nucleation and subsequent crystallization or aggregation. This technique uses a coherent light source to study the movement of small scattering particles within a liquid suspension. The time constant for the “speckle” at a given scattering angle is directly proportional to the diffusion constant for the scattering medium. This in turn is inversely proportional to the hydrodynamic radius of the scattering particles. Thus, a measurement of the speckle time constant can yield a direct measurement of the average particle size within the protein solution.

Traditional DLS analysis tools utilize fixed (large) angle detectors with high quality optics. However, small angle scattering has been shown to be effective in measuring the diffusion constant. Since more light may be collected at small angles, lower cost optics and detectors may be used. A more detailed diagram of the optics is shown in FIG. 9. The technique uses a laser beam 35, a scattering sample 31, some simple optics to collect scattered light 33A, 33B, a beam stop 36 to remove unscattered light 35, and a CCD camera 34 to record the data. The optics 33 are employed to direct the scattered light 32 on to the CCD imager 34, which digitizes the light intensity. The resulting image is analyzed for scattering and time varying correlations to determine the SLS and DLS results. In an alternative embodiment, the CCD may be replaced with a low cost CMOS sensor.

In operation, in an exemplary embodiment, initial conditions are chosen for the crystallization experiments according to the solubility map that was generated, i.e, data in the database 12. Nanoliters of motherliquid at the chosen conditions are loaded in the crystallization chamber 30. The reservoirs may contain a drying agent(e.g., air, or saltwater), humidifying agent(e.g. water), acid and base, and the like. The computer program 16 allows for fluids to be directed into the chamber 30. Initially, the mother liquid shrinks by dry air to form crystal seed. Through dynamic light scattering, the computer will detect the growth of the protein crystal seeds. If numerous micro seeds are detected, the humidifying agent directed into the chamber 30 in order to dissolve the smaller seeds by expanding the droplet 31 of mother liquid. As less crystal seeds are left, experimental conditions can be refined further. In-situ pH conditions are designed to be changed since pH is another important factor effecting the quality of the protein crystals. For example, as the acid is directed into the chamber 30, the acidity of the droplet 31 ought to increase which can be indicated by a pH indication solution. If the researcher decided to lower the acidity, a base solution can be directed into the chamber 30 by the controller 14 instead. Temperature is monitored at all times by the implanted thermistors 38.

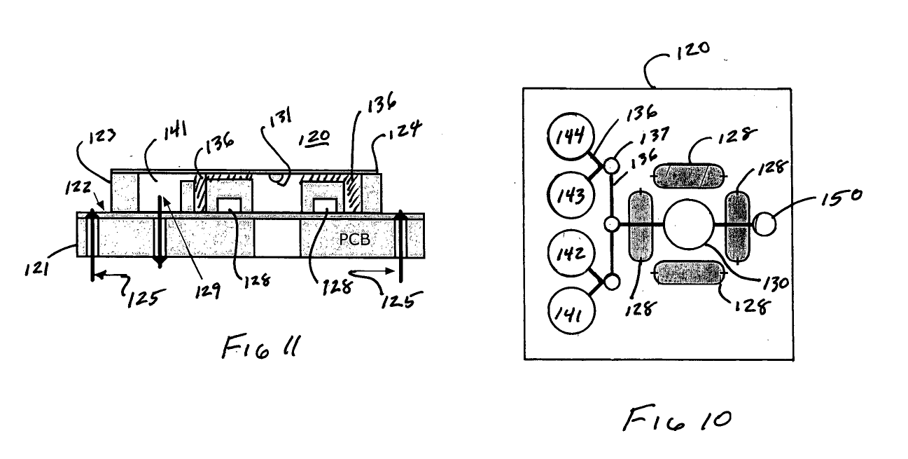

A schematic of another embodiment of the crystallization platform 120 is shown in FIGS. 10 and 11. The platform 120 preferably comprises a PCB bottom layer 121, a plastic middle layer 123 containing reservoirs 141-144 for one or more solutions and an air exit port 150, and a PDMS isolation layer 122 between the PCB layer 121 and the plastic layer 123. A pair of electrodes 129 is placed in each reservoir 141-144. The reservoirs 141-144 are connected by small tubes 136 to a chamber 130 which contains a volume of solution 131 for the purpose of performing a crystallization experiment. By generating bubbles in the reservoir (through electrolysis or boiling), humid gas will move from the reservoir 141-144 to the crystallization chamber 130. Some of the chemical species in this gas will permeate into the crystallization drop 131 and change its chemical property. This can change the water content, pH, or other property, depending on the type of solution that was in the reservoir. The platform 120 may contain small heaters and thermal sensors 128 integrated on the platform 120 to control temperature, and optical ports for optical analysis (e.g., scattering, absorbance, image, etc.).

The platform or chip 120 is designed to fit using a plurality of pins 125 into a motherboard assembly that provides power and control over the vapor process and the temperature circuitry. The chip 120 uses a disposable cover-slip 124 to hold the protein solution 131, so the chip 120 may be re-used if desired. The microcrystallization chip 120 seats into the instrument using standard IC header sockets, and is aligned under a microscope objective (not shown) that is instrumented to a CMOS image sensor (not shown). The platform 120 also contains electronics to control the current through each reservoir, provide illumination for the imager, monitor current, monitor temperature, and communicate with the controller or other host computer.

The controller 14 preferably runs the software 16 that controls and monitors the chip 120 and takes image data at predetermined intervals for time-lapse analysis and monitoring. The software may be controlled and monitored remotely through custom web-interface software. The software and instrument enables a researcher to perform nanodrop crystallization studies from a completely remote location such as home or office.

In operation, in another exemplary embodiment, initial conditions are chosen for the dynamic vapor control experiment according to the solubility map of the compound or protein of interest. Nanoliters of mother liquid at the chosen conditions are loaded in the crystallization chamber 130. The four reservoirs 141-144 preferably contain a drying agent(e.g., air, or saltwater), humidifying agent(e.g. water), acid and base respectively. The computer program 16 allows any of the four reservoirs to be turned on by applied current through the platinum electrodes 129. Initially, the mother liquid shrinks by dry air to form crystal seed. Through dynamic light scattering, the controller 14 will detect the growth of the protein crystal seeds. If numerous micro seeds are detected, the humidifying agent is turned on instead in order to dissolve the smaller seeds by expanding the droplet 131. As less crystal seeds are left, experimental conditions can be refined further. In-situ pH conditions are designed to be changed since pH is another important factor effecting the quality of the protein crystals. For example, as the acid reservoir is turned on, the acidity of the droplet 131 ought to increase which can be indicated by a pH indication solution. If the researcher decides to lower the acidity, the base reservoir can be switched on by the controller. Temperature is monitored at all times by the implanted thermistor 128.

In the foregoing specification, the invention has been described with reference to specific embodiments thereof. It will, however, be evident that various modifications and changes may be made thereto without departing from the broader spirit and scope of the invention. For example, the reader is to understand that the specific ordering and combination of process actions shown in the process flow diagrams described herein is merely illustrative, unless otherwise stated, and the invention can be performed using different or additional process actions, or a different combination or ordering of process actions. As another example, each feature of one embodiment can be mixed and matched with other features shown in other embodiments. Features and processes known to those of ordinary skill may similarly be incorporated as desired. Additionally and obviously, features may be added or subtracted as desired. Accordingly, the invention is not to be restricted except in light of the attached claims and their equivalents.

What is claimed is:

1. A crystallization system comprising
a database comprising quantitative information about the chemical and physical properties under various thermodynamics conditions of one or more compounds, and
a feedback assisted, dynamically controlled chemical system coupled to the database.

2. The system of claim 1, wherein the chemical system comprises
a controller that accesses the database and can monitor and control the state of one or more experiments, and
a crystallization platform coupled to the controller.

3. A crystallization system comprising
a crystallization device,
a controller coupled to the crystallization device, the controller including a memory comprising
a protein solubility database, and
a software program adapted to access the database.
